(19) United States
(12) Patent Application Publication
(10) Pub. No.: US 2011/0240757 A1
(43) Pub. Date: Oct. 6, 2011

(54) Floating UV Fountain

(75) Inventors: Michael Selk, Rockaway, NJ (US); Raymund Vivar, Palm Beach Gardens, FL (US)

(73) Assignee: GMJ DESIGN GROUP, LLC, Lake Worth, FL (US)

(21) Appl. No.: 12/753,443

(22) Filed: Apr. 2, 2010

(51) Int. Cl. B05B 17/08; F21S 8/00

(52) U.S. Cl. 239/18; 239/20

(57) ABSTRACT

A floating fountain with internal ultraviolet water treatment, which comprises a chamber adapted to float near the water surface in a water environment, a water inlet on the top of the chamber for receiving surface water from the water environment, a pump inside the chamber for receiving water from the water inlet, an ultraviolet source for treating water; and a water outlet nozzle at the top of the chamber for receiving water which has been treated with ultraviolet energy and pumped, to provide a pumped-water stream above the water surface.

### FIELD OF THE INVENTION

The present invention relates to fountains for use in ponds or swimming pools.

### BACKGROUND OF THE INVENTION

Fountains have been used for swimming pools and ponds. The fountains shoot water up in a stream for visual effect. Fountains are typically at the bottom of a pool or pond, and a tube connects the pump to a fountain head above the water surface.

### SUMMARY OF THE INVENTION

The present invention provides a floating fountain for use in swimming pools and ponds, which has lights and a UV (ultraviolet) clarifier for cleaning the algae in the water. The algae typically are concentrated on the water surface. By collecting water at the top surface, and treating the water with a UV clarifier, the fountain provides an ornamentally-pleasing fountain pattern in which the water is cleaned. Also, water becomes aerated, which is beneficial for fish.

The present invention provides a floating fountain with internal ultraviolet water treatment, comprising a chamber adapted to float near the water surface in a water environment, a water inlet on the top of the chamber for receiving surface water from the water environment, a pump inside the chamber for receiving water from the water inlet, an ultraviolet source for treating water; and a water outlet nozzle at the top of the chamber for receiving water which has been treated with ultraviolet energy and pumped, to provide a pumped-water stream above the water surface.

The invention provides a floating fountain with internal ultraviolet water treatment, comprising a chamber adapted to float near the water surface in a water environment, the chamber having a screen at the top, a water inlet on the top of the chamber for receiving surface water from the water environment, a pump inside the chamber for receiving water from the water inlet, said pump having a filter, an ultraviolet source for treating water, a water outlet nozzle at the top of the chamber extending above the screen for receiving water which has been treated with ultraviolet energy and pumped, to provide a pumped-water stream above the water surface, and at least one light source to direct light upwards to illuminate the pumped-water stream.

### BRIEF DESCRIPTION OF THE DRAWINGS

FIG. 1 is a perspective exploded view of the UV fountain, according to the invention;

FIG. 2 is a perspective side view, partially broken away, of the UV fountain, according to the invention;

FIG. 3 is a perspective view, partially broken away, of the outside chamber of the UV fountain;

FIG. 4 is a bottom perspective view of the UV fountain; and

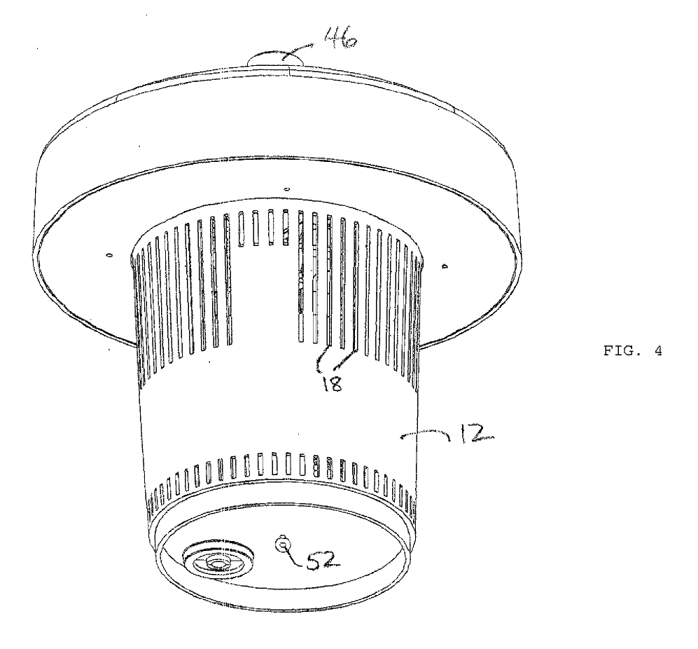

FIG. 5 is a perspective side view, partially broken away, showing internal wiring connections.

### DETAILED DESCRIPTION OF THE PREFERRED EMBODIMENT

A preferred embodiment will be described, but the invention is not limited to this embodiment.

The present invention provides a floating fountain with internal ultraviolet water treatment, comprising a chamber adapted to float near the water surface in a water environment, a water inlet on the top of the chamber for receiving surface water from the water environment, a pump inside the chamber for receiving water from the water inlet, an ultraviolet source for treating water; and a water outlet nozzle at the top of the chamber for receiving water which has been treated with ultraviolet energy and pumped, to provide a pumped-water stream above the water surface.

The chamber may have a screen at the top. The water outlet nozzle may extend above the flat screen. The fountain may further include at least one light source. The fountain may further include a filter. The chamber may have a buoyancy ring around the top screen. The buoyancy of the buoyancy ring may be such that the water inlet is submerged below the water surface when placed in a water environment. The fountain may include an anchor mount at the bottom of the chamber. The fountain may further include a ballast to maintain the chamber in a selected orientation in a water environment.

The invention provides a floating fountain with internal ultraviolet water treatment, comprising a chamber adapted to float near the water surface in a water environment, the chamber having a screen at the top, a water inlet on the top of the chamber for receiving surface water from the water environment, a pump inside the chamber for receiving water from the water inlet, said pump having a filter, an ultraviolet source for treating water, a water outlet nozzle at the top of the chamber extending above the screen for receiving water which has been treated with ultraviolet energy and pumped, to provide a pumped-water stream above the water surface, and at least one light source to direct light upwards to illuminate the pumped-water stream.

The buoyancy of the buoyancy ring may be such that the water inlet is submerged below the water surface when placed in a water environment. The fountain may further include an anchor mount at the bottom of the chamber. The fountain may further include a ballast to maintain the chamber in a selected orientation in a water environment.

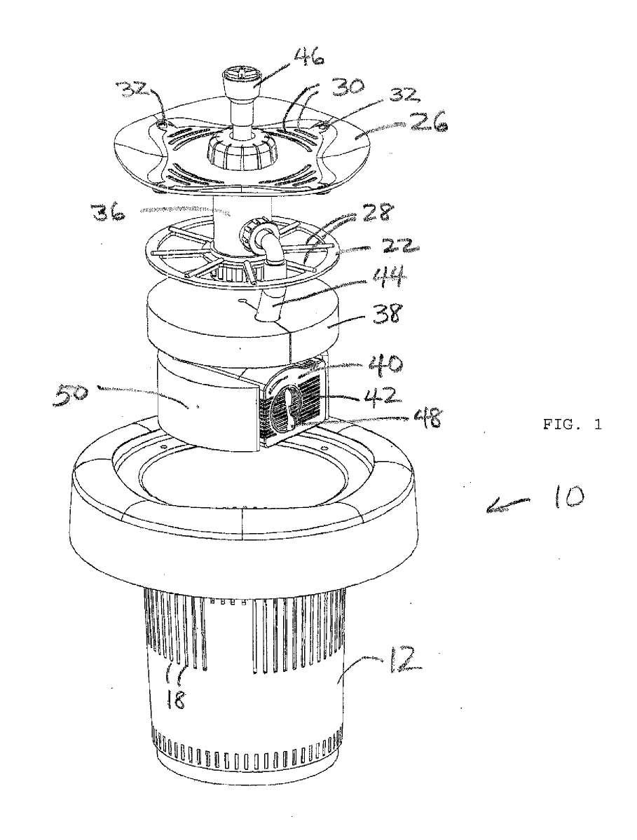

FIG. 1 shows a perspective view of a UV (ultraviolet) fountain 10, according to the invention. The fountain 10 comprises a chamber 12, around the top of which is a buoyancy ring chamber 14 filled with buoyancy material 16. The material type and amount are selected so that the fountain top is preferably just below the surface of the water in which it is placed, e.g., 0.5 inches down. The chamber 12 has a generally-cylindrical shape with downwardly sloping side walls, and vertical chamber slits 18 around its circumferential side walls.

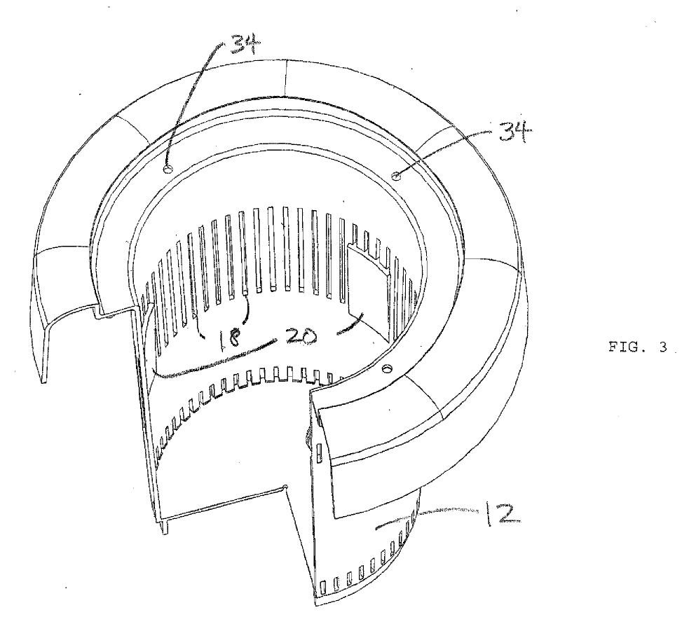

As seen in FIG. 3, the inside of the chamber 12 has ledge members 20 to support a lower bracket 22. The lower bracket 22 has reinforcing lower bracket ribs 28. At the top of the chamber is a top screen 26, having a wavy-shape, arcuate inlet slots 30, and holes 32 through which light from LEDs 34 can be projected upward. The screen serves to collect large material, such as leaves.

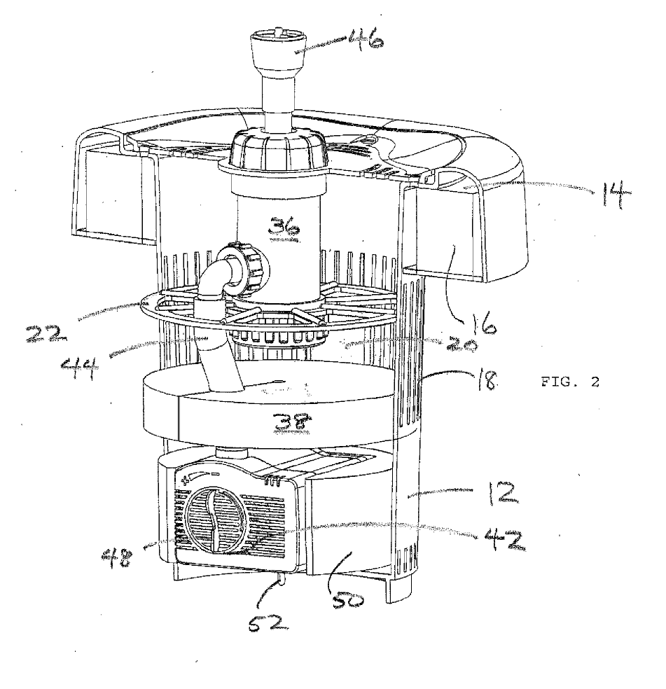

As seen in FIG. 2, a UV clarifier water treatment unit 36 is provided to sanitize the algae that may be present in the water. Below the UV clarifier 36 and lower bracket 22, is a filter pad 38, and below that, a pump 40.

The pump 40 has an inlet 42 and a pump outlet 44. Generally, the water enters the chamber at the top, through screen 26, passes through filter pad 38, and then into pump inlet 42 into the pump 40. The pump 40 then pumps the water up through the UV clarifier 36 and then out the top through a nozzle head 46 which preferably has a plurality of openings or nozzles in a fountain head arranged so that the water streams up above the water surface to provide an ornamentally-attractive spray pattern. The pump 40 has a knob 48 to adjust the speed throughput of the pump.

An access door may be provided in the chamber to enable access to the inside components for service and maintenance. As shown in FIG. 2, the filter pad 38 has a radial slit so that it can be removed from above the pump 40 for cleaning or replacement, without having to disconnect the tubing or cords to the pump.

The pump may have a foam block 50, as shown in FIGS. 1 and 2 to protect the pump during shipment and handling, and possibly during use.

The plurality of LED lights 34 are preferably arranged around the top of the chamber so that the fountain and its spray pattern can be seen in low, ambient light conditions. A power cord to the pump and other components provides power, which can be low-voltage or regular 110 V AC.

Ballast may be provided to stabilize the chamber and fountain during use. The pump itself may provide sufficient ballast, as it is located at the bottom of the chamber. An anchoring ring 52 is provided to which a cord could be attached to keep the device within a certain location or area.

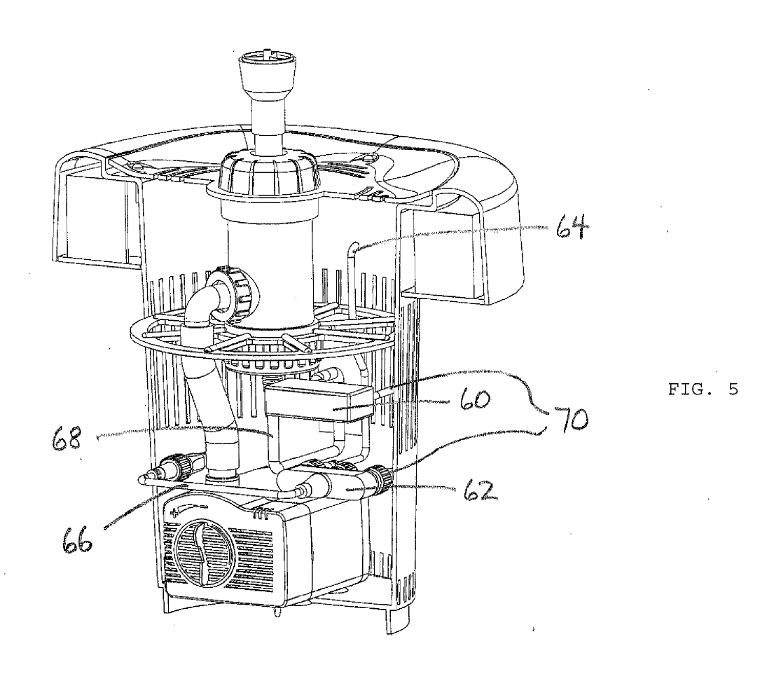

FIG. 5 shows the internal wiring, including an electrical ballast 60 for the UV, a splitter 62, a wiring lead 64 for the LEDs, a wire 66 from the pump 40, or wire 68 for the UV unit (after the ballast, and a wire 70 from the pump 40 to the UV ballast.

Although a preferred embodiment has been described, the invention is not limited to this embodiment, and is defined only by way of the following claims.

What is claimed is:

1. A floating fountain with internal ultraviolet water treatment, comprising:
a chamber adapted to float near the water surface in a water environment;
a water inlet on the top of the chamber for receiving surface water from the water environment;
a pump inside the chamber for receiving water from the water inlet;
an ultraviolet source for treating water; and
a water outlet nozzle at the top of the chamber for receiving water which has been treated with ultraviolet energy and pumped, to provide a pumped-water stream above the water surface.

2. The fountain according to claim 1, wherein the chamber has a screen at the top.

3. The fountain according to claim 2, wherein the water outlet nozzle extends above the flat screen.

4. The fountain according to claim 1, further including at least one light source.

5. The fountain according to claim 1, further including a filter.

6. The fountain according to claim 1, wherein the chamber has a buoyancy ring around the top screen.

7. The fountain according to claim 6, wherein the buoyancy of the buoyancy ring is such that the water inlet is submerged below the water surface when placed in a water environment.

8. The fountain according to claim 1, further including an anchor mount at the bottom of the chamber.

9. The fountain according to claim 1, further including a ballast to maintain the chamber in a selected orientation in a water environment.

10. A floating fountain with internal ultraviolet water treatment, comprising:
a chamber adapted to float near the water surface in a water environment, the chamber having a screen at the top;
a water inlet on the top of the chamber for receiving surface water from the water environment;
a pump inside the chamber for receiving water from the water inlet, said pump having a filter;
an ultraviolet source for treating water;
a water outlet nozzle at the top of the chamber extending above the screen for receiving water which has been treated with ultraviolet energy and pumped, to provide a pumped-water stream above the water surface; and
at least one light source to direct light upwards to illuminate the pumped-water stream.

11. The fountain according to claim 10, wherein the buoyancy of the buoyancy ring is such that the water inlet is submerged below the water surface when placed in a water environment.

12. The fountain according to claim 10, further including an anchor mount at the bottom of the chamber.

13. The fountain according to claim 10, further including a ballast to maintain the chamber in a selected orientation in a water environment.
